(19) United States
(12) Patent Application Publication
(10) Pub. No.: US 2012/0259349 A1
(43) Pub. Date: Oct. 11, 2012

(54) SURGICAL DEVICE FOR MEMBRANE DELIVERY

(75) Inventors: Timothy R. Brahm, Germantown, TN (US); Kevin Foley, Germantown, TN (US)

(73) Assignee: BioD, LLC, Cordova, TN (US)

(21) Appl. No.: 13/441,960

(22) Filed: Apr. 9, 2012

Related U.S. Application Data
(60) Provisional application No. 61/473,272, filed on Apr. 8, 2011.

(51) Int. Cl. A61B 17/03

(52) U.S. Cl. 606/151

(57) ABSTRACT

A surgical device for the delivery of a membrane or other material to a surgical site is provided.

### CROSS-REFERENCE TO RELATED APPLICATIONS

The present application claims priority to U.S. provisional application No. 61/473,272 filed Apr. 8, 2011.

### FIELD OF THE INVENTION

The present invention relates, in general, to a surgical instrument and, more particularly, to a mechanism to deliver a membrane to a surgical site.

### BACKGROUND OF THE INVENTION

In laparoscopic and other minimally invasive surgery, long shafted instruments are used through trocar tubes to grasp, cut, dissect, and manipulate tissue, internal organs or other body parts during the surgical procedure. Instruments for use in laparoscopic, endoscopic and minimally invasive surgery generally have a handle external to the body cavity for controlling an end effector within the body cavity and an elongated shaft including a mechanism for transferring motion and force to the end effector. In such instruments, the end effector is generally a tool on the distal end of the coaxial shaft for cutting, grasping, or dissecting tissue. In addition, a coaxial shaft can be provided to make the instrument generally rotatable with respect to the handle.

In spinal surgery, such as lumber spinal surgery, frequently the surgery is done laproscopically through a trocar tube to facilitate the use of a minimally invasive technique to reduce post-operative recovery time. In this instance, or any other surgical procedure in which the formation of scar tissue or fibrosis may increase the likelihood of post-operative complications, a thin flexible membrane may be placed over the dura, nerve roots, internal organs or implanted surgical hardware to act as an in vivo adhesion barrier or wound covering prior to closing the incision. Given the flexible nature of the membrane and the small surgical opening indicative of a minimally invasive procedure, the membrane can be very difficult for the surgeon to properly place at the surgical site. Specifically, the membrane often adheres to itself or the surgical instruments as the surgeon is attempting to place it in vivo or, in many instances, the membrane is simply too large to fit through the incision without folding or rolling the membrane. In each instance, the challenges associated with the proper placement of the membrane is frustrating for the surgeon and often requires additional time to complete the procedure or failure to use the membrane thereby impacting patient outcomes associated with post-operative scar tissue.

### SUMMARY OF THE INVENTION

The present invention is directed to a surgical instrument for the delivery of a membrane or other material to a surgical site. In one aspect, a surgical device is provided that includes a disposable tip having a proximal and distal end, a holding member attached to a distal end of the disposable tip, an air regulator positioned at a proximal end of the disposable tip, and a first suction tube positioned within the air regulator. The first suction tube is capable of engaging a second suction tube attached to an air pump and is in air flow communication with a plurality of suction channels that traverse the removable tip. The holding member includes a mesh material capable of allowing air flow. The membrane is at least one an adhesion barrier, amniotic tissue wound covering, graft, topical hemostat, surgical mesh, or surgical patch. The air regulator further comprises a knob member for adjusting or stopping air flow.

According to another aspect, a surgical device is provided that includes a holding member configured to retain a membrane to a bottom surface of the holding member, a dispensing member disposed on the holding member and positioned to dispense the membrane from the holding member to a surgical site, and a rod member having a proximal end engaged with a trigger within a handle and a distal end engaged with the dispensing member. The membrane is at least one an adhesion barrier, amniotic tissue wound covering, graft, topical hemostat, surgical mesh, or surgical patch. The membrane comprises a sterile liquid (e.g., a wet graft). When the trigger is engaged, the rod member moves from a first, resting position to a second, engaged position thereby moving the dispensing member from a first position to a second position extending beyond the bottom surface of the holding member. Thus, when the trigger is engaged, the membrane is forced off of the holding member by the dispensing member and released to a surgical site.

According to another aspect, a surgical device is provided that includes a dispensing member positioned to dispense a membrane to a surgical site and a plurality of tines disposed within the dispensing member having proximal ends engaged with a ring member and a distal member engaged with a plurality of attaching members. The ring member is engaged with a trigger within a handle via a rod member. The membrane is at least one an adhesion barrier, amniotic tissue wound covering, graft, topical hemostat, surgical mesh, or surgical patch. When the trigger is engaged, the ring member moves from a first, resting position to a second, engaged position via movement of the rod member and thereby moves the plurality of tines from a first position extending beyond a bottom surface of the dispensing member to a second position retracted above a bottom surface of the dispensing member. Each attaching member is capable of adhering to or piercing the membrane when the plurality of tines is at the first position. Thus, when the trigger is engaged, the membrane is forced off the plurality of attaching members by the dispensing member and released to a surgical site.

### BRIEF DESCRIPTION OF THE DRAWINGS

The novel features of the invention are set forth with particularity in the appended claims. The invention itself, however, both as to organization and methods of operation, together with further objects and advantages thereof, may best be understood by reference to the following description, taken in conjunction with the accompanying drawings in which:

FIG. 1 is a perspective view of one embodiment of the device according to the present invention.

FIG. 2A is a bottom view of an embodiment of the holding member according to the device of FIG. 1.

FIG. 2B is a perspective view an alternative embodiment of the device of FIG. 1.

FIG. 3 is a bottom view of one embodiment of the holding member according to the device of FIG. 1.

FIG. 4 is a perspective view of one embodiment of the device according to the present invention.

FIG. 5 is a view of the bottom of the holding member and dispensing member according to one embodiment.

FIG. 6 is a perspective view of another embodiment of the device according to the present invention.

FIG. 7 is a perspective view of another embodiment of the device according to the present invention.

### DETAILED DESCRIPTION OF THE INVENTION

A device for the delivery of a membrane or other material to a surgical site is provided. As referred to herein, the term “membrane” includes any sheet of surgical material such as an adhesion barrier or other types of fabrics or grafts used in surgery, such as, for example, amniotic tissue wound coverings or grafts, topical hemostats, surgical meshes and surgical patches.

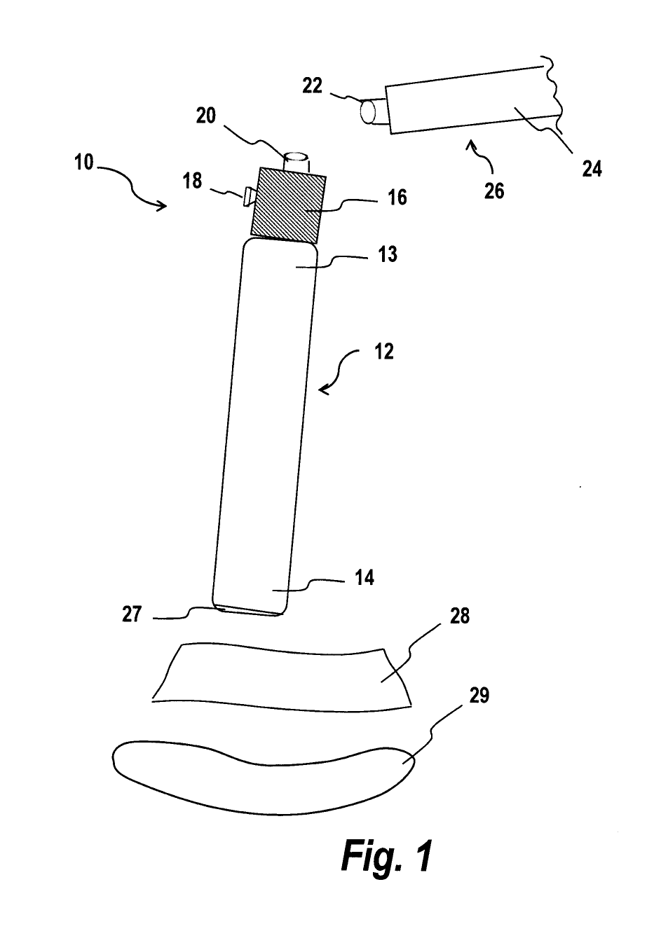

FIG. 1 illustrates one embodiment of a device 10 according to one embodiment. The device 10 includes a disposable tip 12 having a proximal end 13 and distal end 14. The disposable tip 12 is manufactured from a recyclable or environmentally friendly material such as a plastic. An air flow regulator 16 is secured in line with the proximal end 13 of the disposable tip 12. The air flow regulator 16 includes a knob 18 for adjusting air flow. A first suction tube 20 is secured to the disposable tip 12 and air flow regulator 16. The first suction tube 20 is adapted to receive or engage a second suction tube 22 located and secured within a sleeve 24 of a suction device 26 which is commonly found in surgical environments. One of ordinary skill appreciates that the sleeve 26 is entirely optional and the second suction tube 22 may stand alone. The suction device 26 is optimally secured to a pump (not shown). In one embodiment, the pump is capable of delivering an air flow in both a forward and reverse direction.

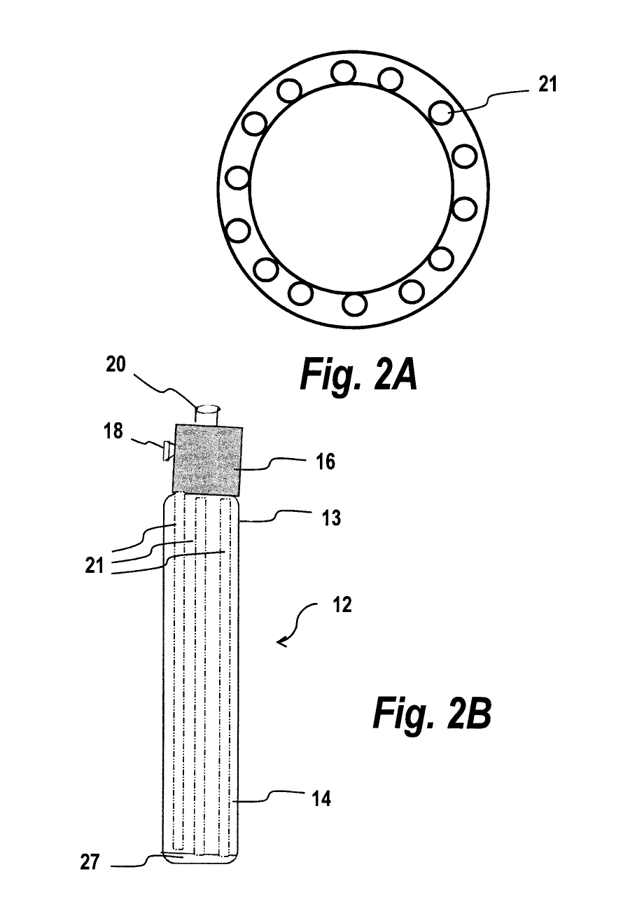

In one embodiment, the first suction tube 20 is secured within and traverses the entire length of the disposable tip 12 and air flow regulator 16. In such an embodiment, the first suction tube 20 terminates at a holding member 27. In another embodiment, as illustrated in FIG. 2A and FIG. 2B, the first suction tube 20 and the air flow regulator 16 are connected to a plurality of suction channels 21 that traverse the entire length of the disposable tip 12 and terminates at the holding member 27. In either embodiment, the holding member 27 is secured to the distal end 14 of the disposable tip 12. The holding member 27 is capable of adhering to a membrane 28 and placing the membrane 28 on a surgical site 29.

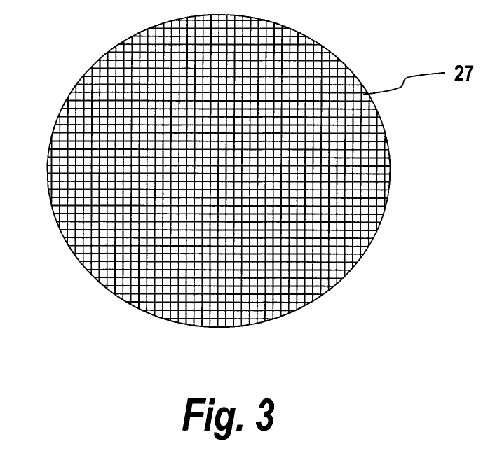

FIG. 3 shows a bottom view of the holding member 27 of the device 10 of FIG. 1. According to this embodiment, the holding member 27 is manufactured from a mesh or screen material or may be manufactured as part of the mold of the device 10. The mesh or screen material can be manufactured from a solid material capable of allowing air to flow either outward or inward to secure and hold the membrane 28 until proper placement and removal. In one embodiment, the mesh or screen is manufactured from a plastic or other polymer-based solid material that is capable of sterile attachment to the membrane 28. In another embodiment, the mesh or screen is manufactured from a metal such as, for example, aluminum, steel, or other metal that is capable of sterile attachment to the membrane 28.

A method for delivering a membrane to a surgical site is provided. According to one embodiment of the method, a user (e.g., surgeon) may utilize the device of FIG. 1 or FIG. 2A/2B to pick up, secure, arrange, and place a membrane 28 over a surgical site 29. To pick up a membrane 28, the user engages the first suction tube 20 with the second suction tube 22. The user turns on the suction device 26 and adjusts the air flow such that air is initially entering through the holding member 27 and flowing through up through the first suction tube 20, through the regulator 16 and to the suction device 26. In one embodiment, the user may adjust the velocity of air flow via the knob 18 on the regulator 16 to an optimal air flow. The optimal air flow is such that the air is moving at a velocity to adhere the membrane 28 to the holding member 27 without resulting in physical damage to the membrane 28. The user contacts the holding member 27 with the membrane 28 to remove the membrane 28 from any packaging that may have held the membrane 28 in a sterile environment. Once positioned on the holding member 27, the user may then move the membrane 28 (while attached to the holding member 27) to the proper surgical site 29. By allowing air to continuously flow, the membrane 28 remains attached while the user attains proper placement over the surgical site 29. Once properly positioned, the membrane 28 is released from the holding member 27 by slowing the air flow or completely stopping air flow via the knob 18 of the regulator 16.

In another embodiment, the second suction tube 22 is removed from the first suction tube 20 and an irrigation bulb (not shown) is attached to the first suction tube 20. The user then delivers a puff of air from the irrigation bulb through the suction tube 20 or plurality of channels 21 and through the holding member 27 to release the membrane 28 from the holding member 27 and onto the surgical site 29.

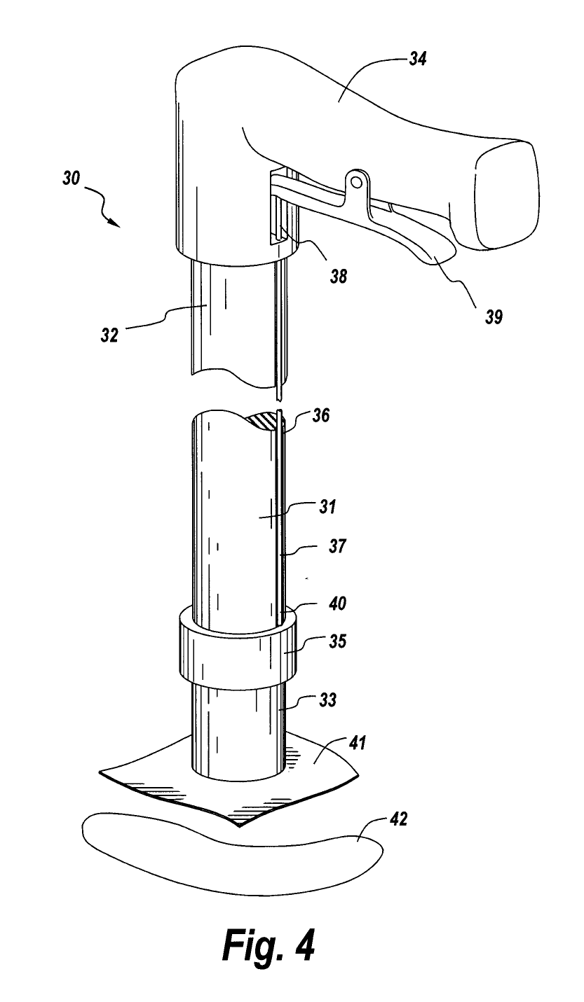

FIG. 4 is a perspective view of another embodiment of the device 30 according to the present invention. According to this embodiment, the device includes a holding member 31 having a proximal end 32 and distal end 33. The proximal end 32 engages and is secured within a handle 34. The distal end 33 engages a dispensing member 35. In the illustrated embodiment, the holding member 31 is rod shaped and the dispensing member 35 is in the shape of a ring that surrounds the holding member 31. The holding member 31 and dispensing member 35 may be manufactured in another functional shape acceptable to movement of the holding member. The holding member 31 and dispensing member 35 may be manufactured from a solid material such as a plastic or metal. The holding member 31 includes a surface defining a channel 36 capable of receiving a rod member 37. The rod member 37 is manufactured from a solid material capable of withstanding force applied by the user such as, for example, a plastic or metal. A first end 38 of the rod member 37 is secured to a trigger 39 located within the handle 34. A second end 40 of the rod member 37 is secured to the dispensing member 35.

A method for delivering a membrane to a surgical site is provided. According to one embodiment of the method, a user (e.g., surgeon) secures her hand on the handle 34 and moves the entire device 30 to position the holding member 32 over a membrane 41 to be placed on a surgical site 42. The user applies enough physical force to the handle 34 to attach the membrane 41 to a bottom surface (see FIG. 4) of the holding member 32. In one embodiment, the membrane 41 is tactile or sticky on either or both a top and bottom surface to aid in adherence to the holding member 32 based on water tension. In such an embodiment, the membrane is preferably a wet membrane (e.g., wet tissue graft) or is a membrane with a wet perimeter. Once the membrane 41 is adhered to the holding member 32, the user moves the device 30 (including the adhered membrane) and positions the holding member 32 over the surgical site 42. The user then places the membrane 41 on the surgical site 42. Once properly placed, the membrane 41 is removed from the holding membrane 32 by the user engaging the trigger 39. When the trigger 39 is engaged, the rod member 37 and dispensing member 35 are, in turn, moved downward towards the membrane 41. Thus, the rod member 37 moves from a first, resting position to a second position where the dispensing member 35 is, in turn, moved from a first, resting position to a second position to touch the membrane 41 thereby releasing the membrane 41 (e.g., forcing the membrane) from the holding member 32. In one embodiment, the rod member 37 is of sufficient length such that, when moved to the second position, the dispensing member 35 is moved slightly beyond the holding member 32 to completely remove the membrane 41 from the bottom surface of the holding member 32.

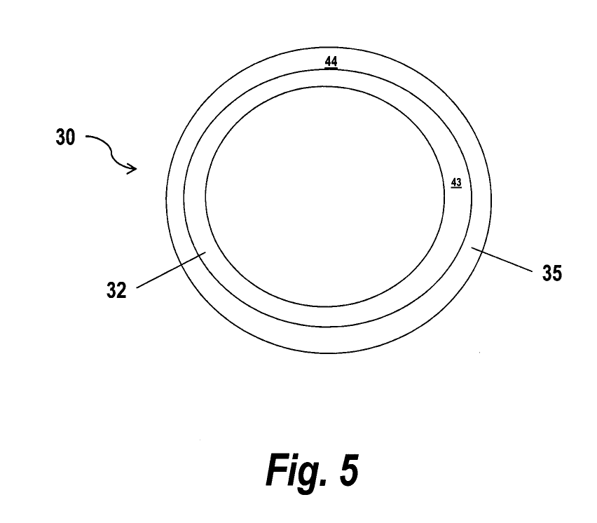

FIG. 5 is a bottom view of the holding member 32 and dispensing member 35 of the device 30. The holding member 32 includes a bottom surface 43 for adhering to the membrane 41. The dispensing member 35 includes a bottom surface 44 for removing the membrane 41 from the bottom surface 44 of the holding member 32. The bottom surface 44 of the dispensing member 35 optionally includes a material that is not susceptible to adherence to the membrane 41. Suitable materials include a plastic or metal that is optionally coated with a material to aid in membrane 41 removal.

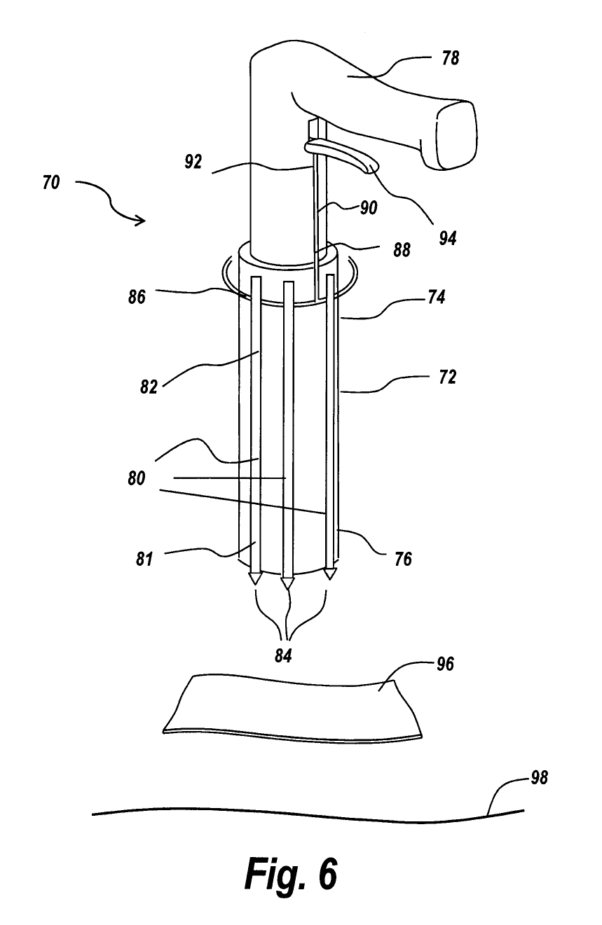

FIG. 6 is a perspective view of another embodiment of the device 70 of the present invention. According to this embodiment, the device 70 includes a dispensing member 72 having a proximal end 74 and distal end 76. The proximal end 74 engages and is secured on a handle 78. A plurality of tines 80 rests on the periphery of the dispensing member 72. Each of the tines 80 includes lower portion 81 and an upper portion 82. The lower portion 81 of each of the tines 80 includes at least one attaching member 84. In one embodiment, the plurality of attaching members 84 extends beyond a bottom surface of the dispensing member 72. The upper portion 82 of each of the tines 80 engages a ring member 86 which holds the plurality of tines 80 against the dispensing member 72 but allows upward and downward movement of the tines 80. The ring member 86, in turn, is secured to a distal end 88 of a rod member 90. A proximal end 92 of the rod member 90 engages a trigger 94 within the handle 78. As illustrated, the dispensing member 72 is rod shaped, however, the dispensing member 72 may manufactured in another functional shape acceptable to allowing movement of the rod member 90 and tines 80. The dispensing member 72, tines 80, and each attaching member 84 may be manufactured from a solid material such as a plastic or metal.

A method for delivering a membrane to a surgical site is provided. According to one embodiment of the method, a user (e.g., surgeon) secures her hand on the handle 78 and moves the entire device 70 to position the attaching members 84 over the membrane 96 to be placed on a surgical site 98. The user applies enough physical force to the handle 78 to attach or pierce the membrane 96 to at least one attaching member 84. In one embodiment, the membrane 96 is tactile or sticky on either or both a top and bottom surface to aid in adherence to at least one tine 84 as well as subsequent movement and placement. Once the membrane 96 is adhered to at least one attaching member 84, the user moves the device 70 (including the adhered membrane) and positions the dispensing member 72 over the surgical site 98 and places the membrane 96 on the surgical site 98. Once properly placed, the membrane 96 is removed from the attaching member 84 by the user engaging the trigger 94. When the trigger 94 is engaged, the rod member 90 is moved upward thereby moving the ring 86 and attached plurality of tines 80 and attaching members 84 upward. Thus, the rod member 90 moves from a first resting position to a second position where the membrane 96 is physically forced off the at least one attaching member 84 by the dispensing member 72.

In alternative embodiment, the membrane 96 may be adhered to a bottom surface of the dispensing member 72 as opposed to the attaching member 84. In such an embodiment, the attaching members 84 are positioned at a point above the bottom surface of the dispensing member 72. Thus, the trigger 94 and rod member 90 are configured to push the tines 80 and attaching members 84 downward thereby pushing the membrane 96 off the dispensing member 72 and on to the surgical site 98.

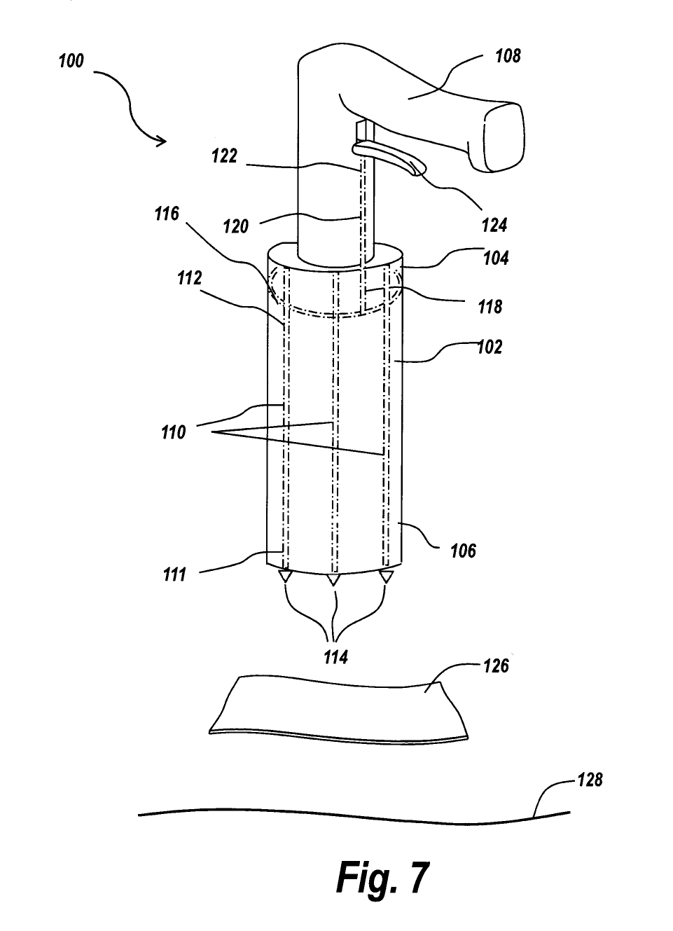

FIG. 7 is a perspective view of a preferred embodiment of the device 100 of the present invention. According to this embodiment, the device 100 includes a dispensing member 102 having a proximal end 104 and distal end 106. The proximal end 102 engages and is secured on a handle 108. A plurality of tines 110 rest inside and substantially traverse the length of the dispensing member 102. Each of the tines 110 includes lower portion 111 and an upper portion 112. The lower portion 111 of each of the tines 110 includes at least one attaching member 114. In one embodiment, the plurality of attaching members 114 extends beyond a bottom surface of the dispensing member 102. The upper portion 112 of each of the tines 110 engages a ring member 116 which holds the plurality of tines 110 against the dispensing member 102 but allows upward and downward movement of the tines 110. The ring member 116, in turn, is secured to a distal end 118 of a rod member 120. A proximal end 122 of the rod member 120 engages a trigger 124 within the handle 108. As illustrated, the dispensing member 102 is rod shaped, however, the dispensing member 102 may manufactured in another functional shape acceptable to allowing movement of the rod member 120 and tines 110. The dispensing member 102 may also have an overall width or circumference that varies according to the site of the target membrane 126. The dispensing member 102, tines 110, and attaching members 114 may be manufactured from a solid material such as a plastic or metal.

A method for delivering a membrane to a surgical site is provided. According to one embodiment of the method, a user (e.g., surgeon) secures her hand on the handle 108 and moves the entire device 100 to position the attaching members 114 over the membrane 126 to be placed on a surgical site 128. The user applies enough physical force to the handle 108 to attach or adhere to the membrane 126 to at least one attaching member 114. In one embodiment, at least one attaching member 114 pierces the membrane 126 to secure the membrane 126 during moving. In such an embodiment, no permanent damage is imposed to the membrane 126 as a result of the piercing. In one embodiment, the membrane 126 is tactile or sticky on either or both a top and bottom surface to aid in adherence to at least one attaching member 114 as well as subsequent movement and placement. Once the membrane 126 is adhered to at least one attaching member 114, the user moves the device 100 (including the adhered membrane) and positions the dispensing member 102 over the surgical site 128 and places the membrane 126 on the surgical site 128. Once properly placed, the membrane 126 is removed from the attaching members 114 by the user engaging the trigger 124. When the trigger 124 is engaged, the rod member 120 is moved upward thereby moving the ring 116 and attached plurality of tines 110 and attaching members 114 upward. Thus, the rod member 120 moves from a first resting position to a second position where the membrane 126 is physically forced off the at least one attaching member 114 by the distal end 106 of the dispensing member 102 as the tines 110 retract within the dispensing member 102.

In an alternative embodiment, the membrane 126 may be adhered to a bottom surface of the dispensing member 102 as opposed to the attaching members 114. In such an embodiment, the attaching members 114 are positioned at a point above the bottom surface of the dispensing member 102. Thus, the trigger 124 and rod member 120 are configured to push the tines 110 and attaching members 114 downward thereby pushing the membrane 126 off the dispensing member 102 and on to the surgical site 128.

While preferred embodiments of the present invention have been shown and described herein, it will be obvious to those skilled in the art that such embodiments are provided by way of example only. Numerous variations, changes, and substitutions will now occur to those skilled in the art without departing from the invention. Accordingly, it is intended that the invention be limited only by the spirit and scope of the appended claims.

1. A surgical device comprising
a disposable tip having a proximal and distal end;
a holding member attached to a distal end of the disposable tip;
an air regulator positioned at a proximal end of the disposable tip; and
a first suction tube positioned within the air regulator,
wherein the first suction tube is capable of engaging a second suction tube attached to an air pump, and
wherein the first suction tube is in air flow communication with a plurality of suction channels that traverse the removable tip.

2. The surgical device of claim 1, wherein the holding member comprises a mesh material capable of allowing air flow.

3. The surgical device of claim 1, wherein the membrane is at least one an adhesion barrier, amniotic tissue wound covering, graft, topical hemostat, surgical mesh, or surgical patch.

4. The surgical device of claim 1, wherein the air regulator further comprises a knob member for adjusting or stopping air flow.

5. A surgical device comprising
a holding member configured to retain a membrane to a bottom surface of the holding member;
a dispensing member disposed on the holding member and positioned to dispense the membrane from the holding member to a surgical site; and
a rod member having a proximal end engaged with a trigger within a handle and a distal end engaged with the dispensing member.

6. The surgical device of claim 5, wherein the membrane is at least one an adhesion barrier, amniotic tissue wound covering, graft, topical hemostat, surgical mesh, or surgical patch.

7. The surgical device of claim 5, wherein the membrane comprises a sterile liquid.

8. The surgical device of claim 5, wherein when the trigger is engaged, the rod member moves from a first, resting position to a second, engaged position thereby moving the dispensing member from a first position to a second position extending beyond the bottom surface of the holding member.

9. The surgical device of claim 8, wherein when the trigger is engaged, the membrane is forced off of the holding member by the dispensing member and released to a surgical site.

10. A surgical device comprising
a dispensing member positioned to dispense a membrane to a surgical site; and
a plurality of tines disposed within the dispensing member having proximal ends engaged with a ring member and a distal member engaged with a plurality of attaching members,
wherein the ring member is engaged with a trigger within a handle via a rod member.

11. The surgical device of claim 10, wherein the membrane is at least one an adhesion barrier, amniotic tissue wound covering, graft, topical hemostat, surgical mesh, or surgical patch.

12. The surgical device of claim 10, wherein when the trigger is engaged, the ring member moves from a first, resting position to a second, engaged position via movement of the rod member and thereby moves the plurality of tines from a first position extending beyond a bottom surface of the dispensing member to a second position retracted above a bottom surface of the dispensing member.

13. The surgical device of claim 12, wherein each attaching member is capable of adhering to or piercing the membrane when the plurality of tines is at the first position.

14. The surgical device of claim 12, wherein when the trigger is engaged, the membrane is forced off the plurality of attaching members by the dispensing member and released to a surgical site.
